(19) United States
(12) Patent Application Publication
(10) Pub. No.: US 2023/0230660 A1
(43) Pub. Date: Jul. 20, 2023

(54) IMAGE GENERATION DEVICE, DISPLAY DEVICE, DATA CONVERSION DEVICE, IMAGE GENERATION METHOD, PRESENTATION METHOD, DATA CONVERSION METHOD, AND PROGRAM

(71) Applicant: Kewpie Corporation, Tokyo (JP)

(72) Inventors: Tomoharu NAGAO, Kanagawa (JP); Shinichi SHIRAKAWA, Kanagawa (JP); Shiori ARII, Kanagawa (JP); Yoshinori KONO, Tokyo (JP); Kurataka OTSUKA, Tokyo (JP); Daisuke KURIKI, Tokyo (JP)

(73) Assignee: Kewpie Corporation, Tokyo (JP)

(21) Appl. No.: 17/996,372

(22) PCT Filed: Apr. 21, 2021
(86) PCT No.: PCT/JP2021/016184

§ 371 Date: Oct. 17, 2022

(30) Foreign Application Priority Data
Apr. 21, 2020 (JP) 2020-075691

(51) Int. Cl. G16B 40/00; G16H 30/20; G06T 7/00; G06V 10/764

(52) U.S. Cl. CPC G16B 40/00; G16H 30/20; G06T 7/0012; G06V 10/764

(57) ABSTRACT

An image generation device includes an imaging unit configured to convert data representing an expression level for each microRNA type into image-rendition data serving as data representing a matrix of two dimensions or more, a classification unit configured to perform classification of the image-rendition data, and a contribution-presentation-image generation unit configured to generate a contribution-presentation image representing a contribution of a specific part of the image-rendition data to the classification.

### TECHNICAL FIELD

The present invention relates to an image generation device, a display device, a data conversion device, an image generation method, a presentation method, a data conversion method, and a program.

The present application claims the benefit of priority on Japanese Patent Application No. 2020-075691 filed on Apr. 21, 2020, the subject matter of which is hereby incorporated herein by reference.

### BACKGROUND ART

Technology for determining the existence/nonexistence of diseases according to expression levels of microRNA has been developed.

For example, Patent Document 1 discloses a morbidity determination device for determining affection of diseases configured to acquire sample data including expression levels for biomarkers including microRNAs. In addition, the morbidity determination device includes a trained model to determine the existence/nonexistence of affection with respect to a plurality of diseases. The morbidity determination device is configured to determine the existence/nonexistence of affection with respect to a plurality of diseases by use of sample data and the trained model.

### CITATION LIST

### Patent Literature Document

Patent Document 1: International Publication WO2018/079840

### SUMMARY OF INVENTION

### Technical Problem

It is preferable not only to determine health conditions regarding the existence/nonexistence of affection based on biomarkers such as microRNA but also to indicate the grounds of determination.

### Solution to Problem

In a first aspect of the present invention, an image generation device includes an imaging unit configured to convert data representing an expression level for each microRNA type into image-rendition data serving as data representing a matrix of two dimensions or more, a classification unit configured to perform classification of the image-rendition data, and a contribution-presentation-image generation unit configured to generate a contribution-presentation image representing a contribution of a specific part of the image-rendition data to the classification.

According to an assignment method for assigning microRNA types to elements of a matrix representing the image-rendition data according to a sequence of five bases through nine bases selected from among nine bases for each microRNA 5′-terminal, the imaging unit may calculate the values of the elements of a matrix representing the image-rendition data based on the expression levels of microRNA types assigned to the elements of the matrix.

The imaging unit may adopt the assignment method for assigning microRNA types to the elements of a matrix according to the Levenshtein distance relating to a sequence of five bases through nine bases selected from among nine bases for each microRNA 5′-terminal.

The image generation device may further include a display unit configured to display the contribution-presentation image generated by the contribution-presentation-image generation unit together with a typical contribution-presentation image in the class selected for the image-rendition data.

The classification unit may classify the image-rendition data into any one of a healthy class, a disease class for each disease, and a pre-symptomatic class for a specific disease provided for at least one disease among diseases.

In a second aspect of the present invention, a display device includes a contribution-presentation-image acquisition unit configured to acquire a contribution-presentation image representing a contribution of a specific part of image-rendition data to classification of the image-rendition data which is produced by converting data representing an expression level for each microRNA type, and a display unit configured to display the contribution-presentation image.

In a third aspect of the present invention, a display device includes a classification unit configured to perform classification of data representing an expression level for each microRNA type, a contribution-presentation-image generation unit configured to generate a grounds-presentation image presenting the grounds of classification in a two-dimensional image, and a display unit configured to display the grounds-presentation image.

In a fourth aspect of the present invention, a data conversion device includes an imaging unit configured to convert data representing an expression level for each microRNA type into image-rendition data, which is represented by a matrix of two dimensions or more and in which microRNA types having a smaller distance defined between index values corresponding to microRNA types are assigned to neighboring elements in the matrix.

According to an assignment method for assigning microRNA types to elements of a matrix representing the image-rendition data according to a sequence of five bases through nine bases selected from among nine bases for each microRNA 5′-terminal, the imaging unit may calculate the values of the elements of a matrix representing the image-rendition data based on the expression levels of microRNA types assigned to the elements of a matrix.

The imaging unit may adopt the assignment method for assigning microRNA types to elements of a matrix according to the Levenshtein distance relating to a sequence of five bases through nine bases selected from among nine bases for each microRNA 5′-terminal.

In a fifth aspect of the present invention, an image generation method includes a process for converting data representing an expression level for each microRNA type into image-rendition data, a process for performing classification of the image-rendition data, and a process for generating a contribution-presentation image representing a contribution of a specific part of the image-rendition data to the classification.

In a sixth aspect of the present invention, a presentation method includes a process for performing classification of data representing an expression level for each microRNA type extracted from an extracted person, a process for generating a grounds-presentation image for presenting the grounds of classification in a two-dimensional image, and a process for displaying the grounds-presentation image to be presented to the extracted person.

In a seventh aspect of the present invention, a data conversion method includes a process for converting data representing an expression level for each microRNA type into image-rendition data, which is represented by a matrix of two dimensions or more and in which microRNA types having a smaller distance defined between index values corresponding to microRNA types are assigned to neighboring elements in a matrix.

In an eight aspect of the present invention, a program causes a computer to implement a process for converting data representing an expression level for each microRNA type into image-rendition data, a process for performing classification of the image-rendition data, and a process for generating a contribution-presentation image representing a contribution of a specific part of the image-rendition data to the classification.

In a ninth aspect of the present invention, a program causes a computer to implement a process for converting data representing an expression level for each microRNA type into image-rendition data, which is represented by a matrix of two dimensions or more and in which microRNA types having a smaller distance defined between index values corresponding to microRNA types are assigned to neighboring elements in the matrix.

### ADVANTAGEOUS EFFECTS OF INVENTION

According to the aforementioned image generation device, the display device, the data conversion device, the image generation method, the presentation method, the data conversion method, and the program, it is possible to indicate the grounds of determination using biomarkers such as microRNAs.

### BRIEF DESCRIPTION OF DRAWINGS

FIG. 1 is a block diagram showing a functional configuration example of an image generation device according to the embodiment.

FIG. 2 is a schematic illustration showing an example of processing expression-level data into a two-dimensional image by an imaging unit according to the embodiment.

FIG. 3 is a block diagram showing a configuration example of a visualizing unit having various parts according to the embodiment.

FIG. 4 is a picture showing a first example of a heatmap on a display unit according to the embodiment.

FIG. 5 is a picture showing a second example of a heatmap on a display unit according to the embodiment.

FIG. 6 is a flowchart showing an example of a procedure to be performed by the image generation device according to the embodiment.

FIG. 7 is a flowchart showing an example of a process to be performed by the visualizing unit according to the embodiment.

FIG. 8 is a block diagram showing a first configuration example of a display device according to another embodiment.

FIG. 9 is a block diagram showing a second configuration example of the display device according to a further embodiment.

FIG. 10 is a block diagram showing a configuration of a computer according to at least one embodiment.

### DESCRIPTION OF EMBODIMENTS

Hereinafter, the present invention will be described by way of embodiments, although the following description of the embodiments does not necessarily limit the scope of the invention defined by claims. In addition, all the combinations of features described in the embodiments are not necessarily essential to the solving means of the present invention.

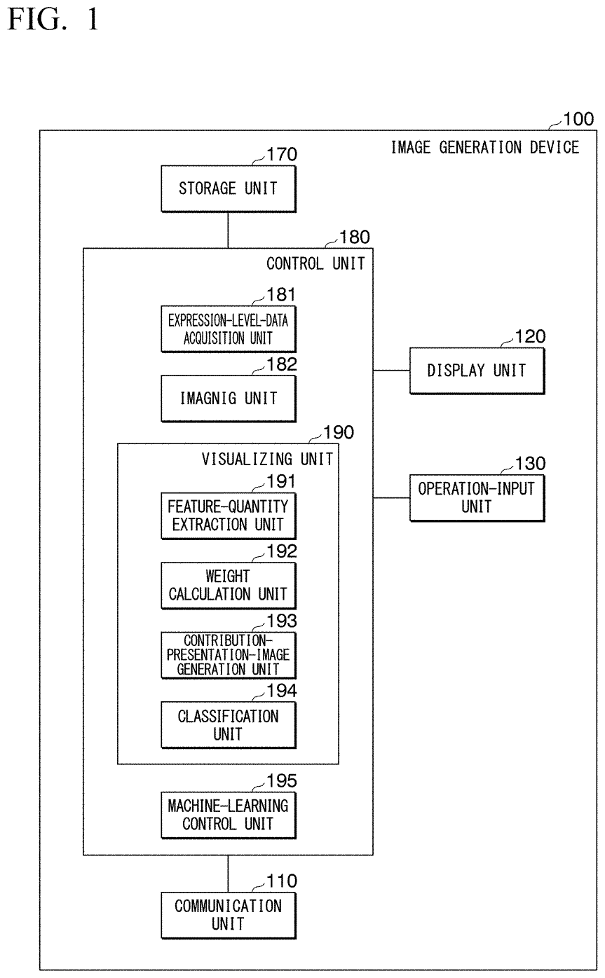

FIG. 1 is a block diagram showing a functional configuration example of an image generation device according to the embodiment. In the configuration shown in FIG. 1, an image generation device 100 includes a communication unit 110, a display unit 120, an operation-input unit 130, a storage unit 170, and a control unit 180. The control unit 180 includes an expression-data acquisition unit 181, an imaging unit 182, and a visualizing unit 190, and a machine-learning unit 195. The visualizing unit 190 includes a feature-quantity extraction unit 191, a weight calculation unit 192, a contribution-presentation-image generation unit 193, and a classification unit 194.

The image generation device 100 is configured to visualize the grounds of classification based on expression-level data of microRNAs (miRNAs). Specifically, the image generation device 100 is configured to extract feature quantity from expression-level data for each type of microRNA. Based on the extracted feature quantity, for example, the image generation device 100 may classify the health condition of a microRNA-extracted person into any one of preset classes such as a normal class, a bladder-cancer class, and a prostatic-cancer class. Subsequently, the image generation device 100 may generate a heatmap showing contribution ratios of expression levels for each type of microRNA due to classification. The heatmap may indicate the grounds of classification since the heatmap can demonstrate which expression level among multiple expression levels for each microRNA is used to perform classification. Hereinafter, the microRNA-extracted person will be simply referred to as an extracted person.

The subject to be handled by the image generation device 100 is not necessarily limited to microRNAs. For example, it is possible to handle various types of subjects, which can be characterized by sequences of elements and which would be measurable in quantity for each sequence (e.g., concentration), such as various types of RNAs or DNAs, protein, and the like. In the case of RNA or DNA, bases may be correspondingly regarded as elements. In the case of protein, amino acids may be correspondingly regarded as elements.

For example, the image generation device 100 can be made up of a personal computer (PC), a work station or the like.

Herein, expression-level data of microRNAs can be defined as data representing an expression level for each type of microRNA. For example, it is said that humans may have more than 2,500 types of microRNAs, wherein when analyzing expression levels for 2,500 types of microRNA, the expression-level data of microRNAs can be expressed by vector data of 2,500 dimensions. For the purpose of acquiring expression-level data of microRNAs, it is possible to use a known sequencing method.

The communication unit 110 is configured to communicate with other devices. For example, the communication unit 110 may communicate with a microRNA-expression-level analysis device to receive expression-level data of microRNAs.

For example, the display unit 120 may be equipped with a display screen such as a liquid-crystal panel and a LED (Light-Emitting Diode) panel, and the like, thus displaying various types of images. For example, the display unit 120 is configured to display results of classification by the image generation device 100 and heatmaps representing the grounds of classification.

For example, the operation-input unit 130 includes an input device such as a keyboard and a mouse to receive user operations. For example, the operation-input unit 130 may receive a user operation to instruct a start of analysis.

The storage unit is configured to store various types of data. The storage unit 170 can be made up of a storage device installed in the image generation device 100.

The control unit 180 is configured to perform various types of functions by controlling various parts of the image generation device 100.

For example, the image generation device 100 may include a CPU (Central Processing Unit) configured to read and execute programs on the storage unit 170, thus achieving the functionality of the control unit 180.

The expression-level-data acquisition unit 181 is configured to acquire expression-level data of microRNAs. Specifically, the expression-level-data acquisition unit 181 may extract expression-level data of microRNA from the reception data from the microRNA-expression-level analysis device via the communication unit 110. Alternatively, it is possible to acquire already-existing expression-level data in such a way that the expression-level-data acquisition unit 181 may simply read expression-level data from the storage unit 170.

The imaging unit 182 is configured to process expression-level data of microRNAs into a two-dimensional image.

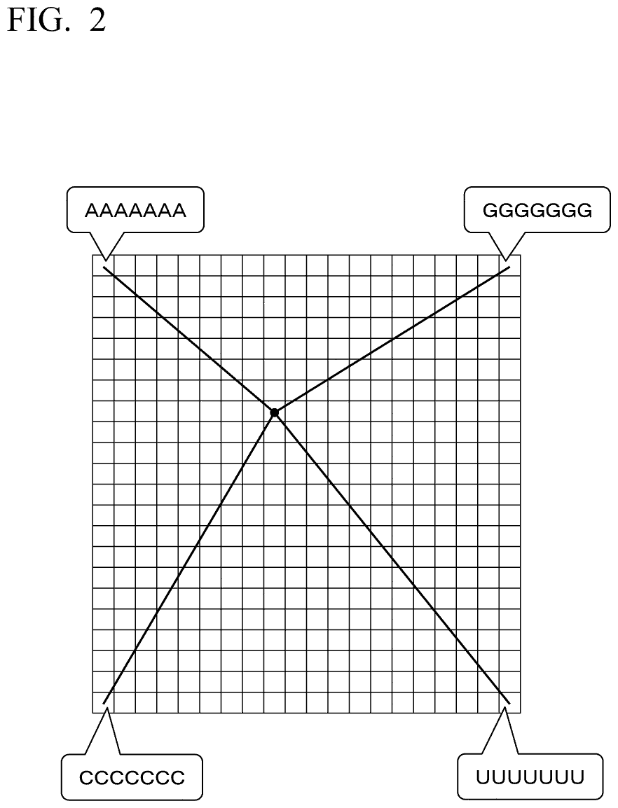

FIG. 2 is a schematic illustration showing an example of processing expression-level data into a two-dimensional image by the imaging unit 182. The imaging unit 182 assigns (or performs mapping) various types of microRNAs represented by expression-level data onto elements of a two-dimensional matrix illustrated in FIG. 2, thus inputting expression levels into elements of a matrix according to assignment.

It is possible to set an arbitrary size of a matrix. The number of elements in a matrix can be approximately set to the same number of dimensions in expression-level data. When the number of dimensions of expression data is set to 2,500, for example, it is possible to use a matrix of 50-row by 50-column or a matrix of 48-row by 48-column.

According to sequences of five bases through nine bases selected from nine bases of the microRNA 5′-terminal, the imaging unit 182 determines assignment of microRNA types into elements of a matrix.

Upon setting seven bases of the mircoRNA 5′-terminal, for example, the imaging unit 182 may specifically calculate a sequence of seven bases of the microRNA 5′-terminal and an alignment of seven adenines as well as the Levenshtein distance. Similarly, the imaging unit 182 calculates a sequence of seven bases of the microRNA 5′-terminal, an alignment of seven guanines, an alignment of seven cytosines, and an alignment of seven uracils as well as the Levenshtein distance.

Using initials of base names, for example, the distance between a base sequence represented by “GAAUCAU” and “AAAAAAA” (i.e., an alignment of seven adenines) will be discussed below. In this case, when the first initial “G”, the fourth initial “U”, the fifth initial “C”, and the seventh initial “U” aligned in the base sequence from its left side are each replaced with “A”, it is possible to convert “GAAUCAU” into “AAAAAAA” with calculating the Levenshtein distance of “4”.

Using a two-dimensional matrix in which the number of rows is equal to the number of columns (i.e., a square matrix), “AAAAAAA”, “GGGGGGG”, “CCCCCCC”, and “UUUUUUU” are assigned to elements at four corners as shown in FIG. 2.

The imaging unit 182 assigns microRNA types to elements of a matrix such that ratios between distances measured from four corners of a two-dimensional matrix may be associated with ratios between Levenshtein distances calculated above. According to the assignment, the imaging unit 182 may convert expression-level data into image-rendition data.

Herein, the image-rendition data can be categorized as data enabling rendition of images, which may be configured as data representing a matrix of two dimensions or more. In this connection, the image-rendition data may be image data, but this is not a restriction. For example, image-rendition data may not conform to prescriptions of specific data formats since image-rendition data may not necessarily be furnished with headers and footers prescribed for specific image data formats.

The number of dimensions of a matrix representing image-rendition data may be equal to the number of dimensions of images subjected to rendition. When an image subjected to rendition is a two-dimensional image, for example, image-rendition data may be realized in the form of a two-dimensional matrix. Alternatively, when an image subjected to rendition is a three-dimensional image, image-rendition data may be realized in the form of a three-dimensional matrix.

In the above, elements of a matrix representing image-rendition data are associated with pixel values of an image subjected to rendition. When an image subjected to rendition is an image of n pixels in its longitudinal direction and n pixels in its lateral direction, for example, image-rendition data may be realized as the data format of a two-dimensional matrix of n-row by n-column. The imaging unit 182 writes expression levels of microRNAs whose types are assigned to elements of a matrix into elements of a matrix in image-rendition data.

The following descriptions exemplarily refer to the case of using image data as image-rendition data, wherein elements of a matrix representing image-rendition data will be denoted as image-data elements.

The imaging unit 182 may determine assignment of microRNA types to pixels according to ratios between distances measured from three corners out of four corners. In the case of FIG. 2, for example, the imaging unit 182 may assign microRNA types to pixels such that ratios between distances measured from three corners assigned “AAAAAAA”, “GGGGGGG”, and “CCCCCCC” can be associated with ratios between Levenshtein distances.

Using ratios of distances measured from three points, it is possible to determine a specific position in a two-dimensional image in a triangulation manner.

Alternatively, the imaging unit 182 may use all the distances measured from four corners to assign microRNA types to pixels. For example, in the case of FIG. 2, the imaging unit 182 may adopt an orthogonal coordinate system using first coordinates passing through the corner assigned “AAAAAAA” and the corner assigned “UUUUUUU” and second coordinates passing through the corner assigned “GGGGGGG” and the corner assigned “CCCCCCC”. Subsequently, the imaging unit 182 may calculate the coordinate values of the first coordinates according to a ratio between the Levenshtein distance for converting the base sequence subjected to conversion into “AAAAAAA” and the Levenshtein distance for converting the base sequence into “UUUUUUU”. Similarly, the imaging unit 182 may calculate the coordinate values of the second coordinates according to a ratio between the Levenshtein distance for converting the base sequence subjected to conversion into “GGGGGGG” and the Levenshtein distance for converting the base sequence into “CCCCCCC”. Upon determining the coordinate values with respect to the first coordinates and the second coordinates, it is possible to determine a specific position in a two-dimensional image.

For the purpose of reviewing characteristics of microRNAs, a sequence of nine bases of the microRNA 5′-terminal is important among bases of twenty or so microRNAs. When the imaging unit 182 assigns microRNA types to pixels according to the Levenshtein distance relating to a sequence of five through nine bases selected from among nine bases of the microRNA 5′-terminal, it is expected that microRNAs having similar characteristics may be disposed at neighboring pixels in a two-dimensional image to be produced.

Although it is preferable that seven bases of the microRNA 5′-terminal be selected for a sequence of bases to be referred to when the imaging unit 182 assigns microRNA types to pixels, it may be sufficient to refer to a sequence of five through nine bases among nine bases of the microRNA 5′-terminal; but this is not a restriction. Responsive to microRNA types used as biomarkers, for example, the imaging unit 182 may select six bases, ranging from the second base to the seventh base in the microRNA 5′-terminal so as to determine mapping between microRNA types and pixels.

The methodology for the imaging unit 182 to assign microRNA types to pixels should not be necessarily limited to the aforementioned method. For example, the imaging unit 182 may adopt an assignment method using the Jaro-Winkler distance instead of the aforementioned Levenshtein distance. Subsequently, the imaging unit 182 may calculate pixel values based on the expression level for each microRNA type.

In this connection, it is possible to assign a plurality of microRNAs to a single pixel. In this case, the imaging unit 182 sums up expression levels of microRNAs to assign its summation to the same pixel to be assigned a plurality of microRNA types.

It is possible to provide unassigned pixels not assigned any microRNA type among pixels. For example, it is possible to set values of unassigned pixels to “0”.

In this connection, expression levels may have negative values. When a certain microRNA type affects something in an inhibitory manner, the microRNA type can be represented by a negative value. On the other hand, due to a possible deviation of expression levels of expression-level data out of a certain range of pixel values, for example, when a two-dimensional image to be produced should have fixed pixel values, e.g., zero or a positive value, the imaging unit 182 may perform normalization to convert expression levels in a certain range of pixel values.

Two-dimensional images (or expression-level data formulized into two-dimensional images) generated by the imaging unit 182 may be referred to as expression-level-data images.

The visualizing unit 190 extracts feature quantity from an expression-level-data image so as to perform classification using the extracted feature quantity. According to the classification, the visualizing unit 190 performs classification with respect to the health condition of an extracted person described above.

In addition, the visualizing unit 190 is configured to generate a heatmap demonstrating the grounds of classification.

The feature-quantity extraction unit 191 is configured to extract feature quantity from an expression-level-data image.

The weight calculation unit 192 is configured to calculate weights representing contributions of pixels in an expression-level-data image with respect to classification.

The contribution-presentation-image generation unit 193 is configured to generate a heatmap demonstrating the grounds of classification. Specifically, the contribution-presentation-image generation unit 193 may generate a heatmap by weighting pixel values of pixels constituting an expression-level-data image with weights calculated by the weight calculation unit 192. As the grounds of classification, the heatmap shows contributions of segments of an expression-level-data image according to classification. Herein, segments of an expression-level-data image may be pixels of an expression-level-data image. An image representing contributions of segments of an input image according to classification will be referred to as a contribution-presentation image.

The following descriptions refer to heatmaps exemplifying contribution-presentation images to be generated by the contribution-presentation-image generation unit 193. In this connection, it is sufficient for the contribution-presentation-image generation unit 193 to generate a contribution-presentation image such as an image showing contributions of segments of an input image according to classification of an input image, which is not necessarily limited to a heatmap.

The classification unit 194 is configured to perform classification of expression-level-data images according to feature quantity extracted by the feature-quantity extraction unit 191. The classification can be interpreted as classification of health conditions of extracted persons according to microRNA types indicated by expression-level data.

The machine-learning control unit 195 is configured to control learning by the visualizing unit 190. For example, the feature-quantity extraction unit 191 and the weight calculation unit 192 can be configured using calculation models such as neural networks. Upon inputting supervised learning data, the machine-learning control unit 195 may control the feature-quantity extraction unit 191 and the weight calculation unit 192 to perform learning to determine parameter values of calculation models.

For example, the process of the visualizing unit 190 and the learning of the visualizing unit 190 can be executed using known techniques for visualizing contributions of segments of images according to image classification such as GCM (Generative Contribution Mappings) and Grad-CAM.

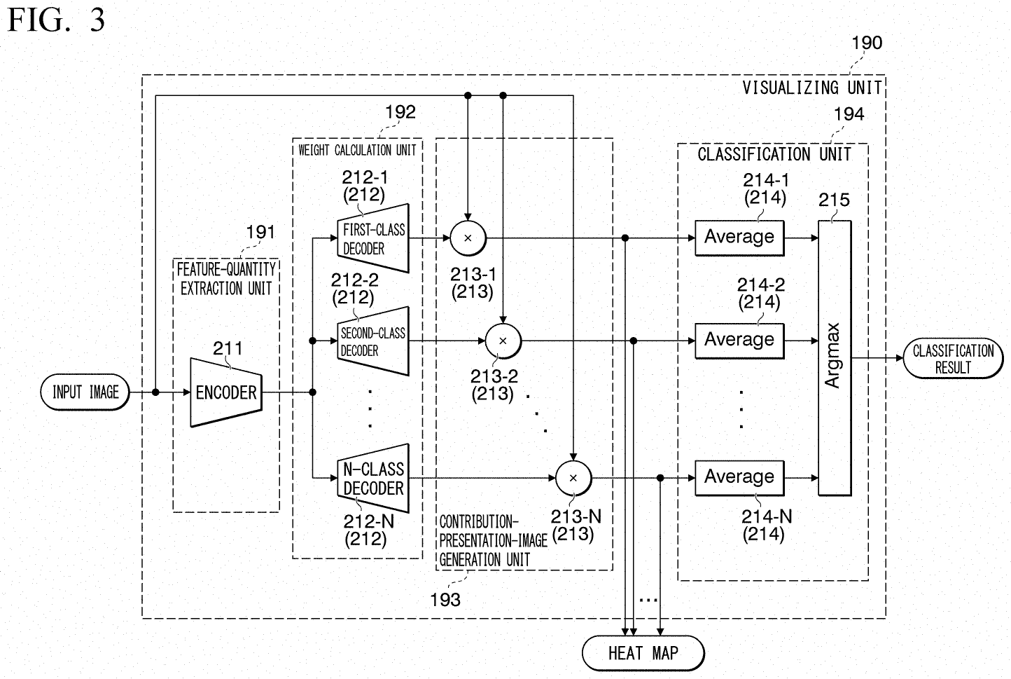

FIG. 3 is a picture showing a configuration example of the visualizing unit 190 having various parts. FIG. 3 shows an example of the visualizing unit 190 to implement its functions using the GCM.

In the configuration shown in FIG. 3, the visualizing unit 190 includes an encoder 211, a first-class decoder 212-1 through an N-class decoder 212-N, a first multiplier 213-1 through an N multiplier 213-N, a first averaging operator 214-1 through an N averaging operator 214-N, and an Argmax operator 215. Herein, “N” denotes an integer showing the number of classes used in classification.

The first-class decoder 212-1 through N-class decoder 212-N will be collectively denoted as decoders 212. The first multiplier 213-1 through the N multiplier 213-N will be collectively referred to as multipliers 213. The first averaging operator 214-1 through the N averaging operator 214-N will be collectively referred to as averaging operators 214.

Upon inputting an image, the encoder 211 extracts feature quantity of the input image. In an example of the image generation device 100, the encoder 211 inputs an expression-level-data to extract its feature quantity.

The encoder 211 may exemplify the feature-quantity extraction unit 191. The decoders 212 are provided for their corresponding classes, wherein the decoder 212 is configured to reconfigure the feature quantity calculated by the encoder 211 into a map having the same number of pixels as the number of pixels in the input image. The map is a weighted map showing weights representing how much various parts of the input image are likely to be suitable for an attentive class. Herein, various parts of the input image may be various pixels of the input image. In this connection, a map calculated by the decoder 212 will be referred to as CWM (Class Weight Map). A combination of the first-class decoder 212-1 through the N-class decoder 212-N may exemplify the weight calculation unit 192.

The multipliers 213 are provided for respective classes, wherein the multiplier 213 is configured to perform multiplication with the CWM, which is calculated for each class by the decoder 212, for each pixel of the input image. Accordingly, it is possible to produce a heatmap showing pixels of the input image weighted according to their contributions to classification. The heatmap calculated by the multipliers 213 will be referred to as CCM (Class Contribution Map).

A combination of the first multiplier 213-1 through the N multiplier 213-N may exemplify the contribution-presentation-image generation unit 193.

The averaging operators 214 are provided for respective classes, wherein the averaging operator 214 is configured to calculate an average of pixel values of the CCM calculated by the multiplier 213 for each class. The average calculated by the averaging operator 214 will be used as an evaluation value in the classification. Herein, the evaluation value may serve as a class score.

The Argmax operator 215 determines a class having the highest value of the class score by comparing class scores calculated by the averaging operators 214 for their respective classes. Thus, it is possible for the Argmax operator 215 to classify the input image for its suitable class.

A combination of the first averaging operator 214-1 through the N averaging operator 214-N as well as the Argmax operator 215 may exemplify the classification unit 194.

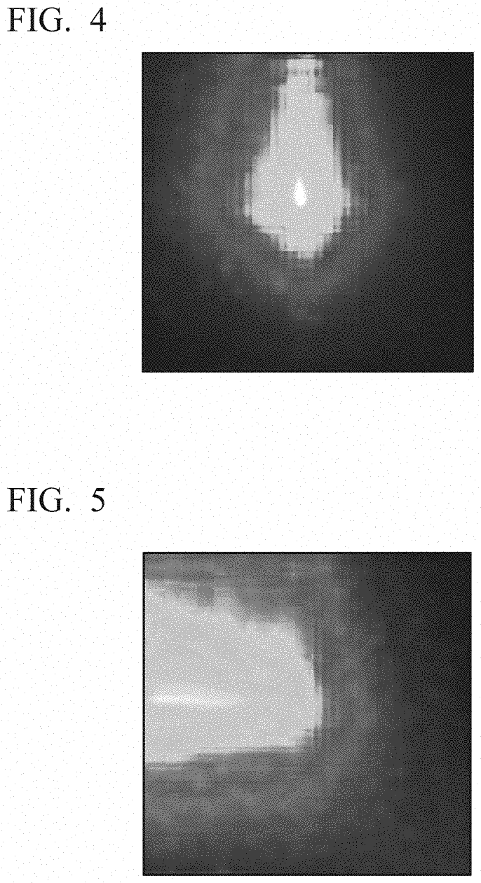

FIG. 4 is a picture showing a first example of a heatmap on the display unit 120.

FIG. 4 shows an example of a heatmap to classify into a healthy class. For example, the display unit 120 is configured to display a heatmap generated by the contribution-presentation-image generation unit 193 under the control of the visualizing unit 190.

As described above, the contribution-presentation-image generation unit 193 is configured to produce a heatmap (CCM) by applying weights responsive to contributions to classes to pixels of an expression-level-data image.

It is possible to set approximately the same magnitude of weights applied to adjacent pixels in the CWM calculated by the decoder 212 by generating an expression-level-data image such that microRNA types having similar characteristics may be disposed at neighboring pixels according to a sequence of five bases through nine bases selected from among nine bases at the microRNA 5′-terminal. Thus, it is possible to produce a heatmap-style image since variations of pixel values between adjacent pixels are controlled to be relatively moderate in an image produced by applying weights to an expression-level-data image.

When a heatmap endures missing of pixels due to the existence of unassigned pixels not assigned any expression level in an expression-level-data image, the imaging unit 182 may process a heatmap in an easy-to-see manner. For example, the imaging unit 182 may perform a process to interpolate pixels in a heatmap. Alternatively, the imaging unit 182 may perform a process to blur images in a heatmap. In this case, it is possible to apply various techniques for eliminating noise in images to the process performed by the imaging unit 182. For example, the imaging unit 182 may use an expansion filter and a contraction filter, alternatively, the imaging unit 182 may use an averaging filter.

FIG. 5 is a picture showing a second example of a heatmap on the display unit 120. FIG. 5 shows an example of a heatmap classified to a cancer class. In an example of FIG. 5, a class for classifying the health condition of a microRNA-extracted person will be referred to as a cancer-A class.

The heatmap of FIG. 5 is different from the heatmap of FIG. 4 in terms of distribution profiles of pixel values and densities of pixel values, wherein the heatmap of FIG. 5 is higher than the heatmap of FIG. 4 in terms of an average of pixel values. When the display unit 120 displays the heatmap of FIG. 4 and the heatmap of FIG. 5 on the screen, a person looking at heatmaps such as an extracted person can grasp the grounds of classification by visually comparing heatmaps. In this connection, the grounds of classification are grounds proving how images are classified into respective classes.

In the above, it is possible to adopt various manners to display heatmaps on the display unit 120 according to the objective or the usage of heatmaps.

For the purpose of demonstrating the grounds of classification with heatmaps, it is possible to display a plurality of heatmaps for multiple classes such that the display unit 120 may display heatmaps for all classes, thus making it possible for any person to visually compare heatmaps for respective classes.

The display unit 120 may display a heatmap of an extracted person as well as heatmaps which are prepared as typical examples of heatmaps for individual classes. Herein, the heatmap of an extracted person is a heatmap which is obtained with respect to an extracted person. Accordingly, any person looking at heatmaps such as an extracted person can determine a matching degree of a match between the heatmap of an extracted person and a typical example of a heatmap. Herein, the matching degree may be similarity.

The display unit 120 may display heatmaps for all classes in which the classification unit 194 classifies expression-level-data images. Alternatively, it is possible to display heatmaps for some classes by displaying heatmaps for only the predetermined classes which are determined in advance as representative classes among multiple classes.

A higher matching degree between the heatmap of an extracted person and typical examples of heatmaps may be considered as a higher accuracy of classification. When the health condition of an extracted person is classified into a disease class, a higher matching degree between the heatmap of an extracted person and typical examples of heatmaps can be estimated as a progression of diseases or a serious state of diseases.

When a heatmap is displayed in various colors, it is possible to display an image portion having a higher contribution to classification in red but to display another image portion having a lower contribution to classification in blue. Thus, it is expected that a heatmap is displayed in red due to the progress of diseases or the serious state of diseases, thus attaching attention of any person looking at heatmaps.

On the other hand, when the health condition of an extracted person is classified into a healthy class, a heatmap partially colored in red may cause a misunderstanding such that the extracted person is currently suffering from diseases. For this reason, when the health condition of an extracted person is classified into a healthy class, the display unit 120 may display images not including red color.

For example, the storage unit 170 may store data of images entirely and uniformly colored in blue. When the classification unit 194 classifies the health condition of an extracted person into a healthy class, the visualizing unit 190 may read data from the storage unit 170 to display an image entirely and uniformly colored in blue on the display unit 120.

Alternatively, the contribution-presentation-image generation unit 193 may generate a heatmap shading in blue; hence, it is possible to generate a heatmap without using red color.

The display unit 120 may display images according to the pre-symptomatic state of a person. Herein, the pre-symptomatic state indicates the state in which a person is not affected with a specific disease but might have subjective symptoms or a person may have an abnormal value when medically examined, precisely indicating a morbid risk of any disease. For example, fatty liver indicates a morbid state with respect to diseases regarding fatty liver, but it may indicate a pre-symptomatic state with respect to diseases regarding liver cancer.

When the classification unit 194 classifies the health condition of an extracted person into a healthy class and any disease class among various disease classes, for example, it is possible to determine a pre-symptomatic state with respect to a disease class having an assessment value more than a predetermined threshold value among non-selected classes. Herein, the assessment value may be a class score.

In this case, the display unit 120 may display a heatmap of an extracted person as well as a typical heatmap for a class to be determined as a pre-symptomatic state. Herein, the typical heatmap may be a typical example of heatmaps. As described above, any person looking at heatmaps such as an extracted person can determine a matching degree between a heatmap of an extracted person and a typical example of a heatmap. Herein, the matching degree may be similarity.

It is possible to further set a pre-symptomatic class in addition to the healthy class and the disease classes. For example, it is possible to set a pre-symptomatic class for a specific disease associated with one disease class among various disease classes. When the classification unit 194 classifies the health condition of an extracted person into a pre-symptomatic class, the display unit 120 may display the heatmap of an extracted person together with a typical heatmap of a pre-symptomatic class. Thus, any person looking at heatmaps such as an extracted person may determine a likelihood of reliably determining a pre-symptomatic state with reference to heatmaps.

In addition, the display unit 120 may display either a typical heatmap of a disease class whose disease is determined at a pre-symptomatic state or a typical heatmap of a healthy class on the screen, or the display unit 120 may display both of them on the screen. Any person looking at heatmaps such as an extracted person may determine whether the heatmap of an extracted person looks close to either the heatmap of a disease class or the heatmap of a healthy class, thus assessing whether the pre-symptomatic state of an extracted person looks relatively close to the disease state or the healthy state.

In this connection, the storage unit 170 may store a history of heatmaps with respect to the same extracted person, and therefore the display unit 120 may display images easy to grasp changes of heatmaps over time under the control of the visualizing unit 190. The history of heatmaps can be produced using heatmaps observed at multiple points of time.

For example, the display unit 120 may display a plurality of heatmaps observed at multiple points of time, which are juxtaposed on the screen. Alternatively, the display 120 may sequentially display heatmaps over a lapse of time such that heatmaps be displayed as moving pictures or heatmaps be displayed in a frame-by-frame advance manner. Herein, an order to sequentially display images in a frame-by-frame advance manner can be defined as an order to sequentially display images by changing images at each predetermined timing.

For example, any person looking at heatmaps such as an extracted person can assess whether his/her disease is progressing or being restored upon grasping whether an occupancy of red portions in a heatmap is increasing or decreasing. In this case, for example, red portions of a heatmap indicate portions having high contribution to classification.

Moreover, the display unit 120 may visually show changes of heatmaps of an extracted person over time comparable to typical heatmaps on the screen such that the heatmap of an extracted person and the typical heatmap are juxtaposed together on the screen or those heatmaps are transparently overlapped with each other.

Any person looking at heatmaps such as an extracted person can assess whether he or she may suffer from progressing diseases or may be recovered from diseases upon grasping whether the heatmap of an extracted person gradually looks similar to the typical heatmap or whether those heatmaps look differently from each other.

The display unit 120 may display a history of heatmaps with respect to pre-symptomatic states.

Any person looking at heatmaps such as an extracted person may necessarily take countermeasures upon grasping any risk leading to diseases with reference to changes of heatmaps over time.

Next, an operation of the image generation device 100 will be described below.

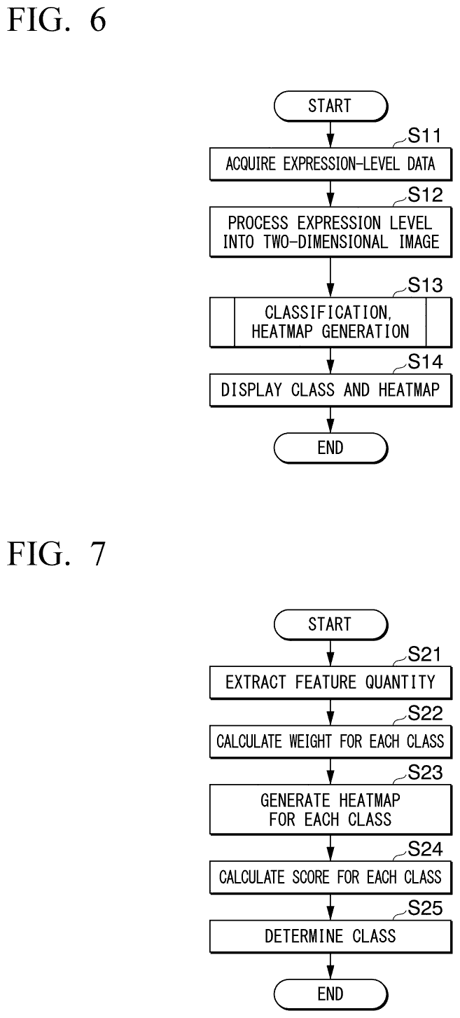

FIG. 6 is a flowchart showing an example of a procedure to be performed by the image generation device 100.

According to the procedure of FIG. 6, the expression-level-data acquisition unit 181 acquires expression-level data (step S11).

Next, the imaging unit 182 converts expression-level data of microRNAs into a two-dimensional image (step S12).

Next, the visualizing unit 190 classifies the health condition of an extracted person into any class while generating a heatmap representing the grounds of classification (step S13).

Subsequently, the display unit 120 displays the heatmap and the result of classification under the control of the visualizing unit 190 (step S14).

After step S14, the image generation device 100 exits the procedure of FIG. 6.

FIG. 7 is a flowchart showing an example of a process to be performed by the visualizing unit 190. The visualizing unit 190 carries out the process of FIG. 7 at step S13 in FIG. 6.

In the process of FIG. 7, the feature-quantity extraction unit 191 extracts feature quantity from an expression-level-data image (step S21).

Next, the weight calculation unit 192 calculates weights for each class representing contributions to pixels in the expression-level-data image in connection with the classification (step S22).

Subsequently, the contribution-presentation-image generation unit 193 applies weights calculated by the weight calculation unit 193 to pixel values of pixels constituting the expression-level-data image, thus generating a heatmap for each class (step S23).

The classification unit 194 calculates an assessment value (or a class score) for each class based on the feature quantity extracted by the feature-quantity extraction unit 191 (step S24).

Based on the calculated class score, the classification unit 194 determines a class for classifying the health condition of an extracted person (step S25).

After step S25, the visualizing unit 190 exits the process of FIG. 7.

As described above, the imaging unit 182 converts data representing an expression level for each microRNA type into image-rendition data. The image-rendition data can be defined as data representing a matrix of two dimensions or more. The classification unit 194 classifies the image-rendition data. The contribution-presentation-image generation unit 193 generates a contribution-presentation image representing a contribution of some part of image-rendition data to classification.

Using the contribution-presentation image, the image generation device 100 can present the grounds of determination (or classification) based on microRNAs. Any person looking at a contribution-presentation image such as an extracted person can determine the likelihood as to whether classification is reliable. When the health condition of an extracted person is classified into a specific disease class, for example, it is possible to assess a degree of progression in diseases or seriousness of diseases upon determining the likelihood as to whether classification is reliable.

In this connection, it is possible to determine the likelihood of classification being accurate based on the size of a specific portion contributing to classification in the entirety of an input image and the magnitude of contribution of the specific portion of the input image. For example, an expression-level image may exemplify the input image. For example, an area ratio may exemplify the size of a specific portion of the input image. For example, a degree of contribution may exemplify the magnitude of contribution.

When the contribution-presentation image is rendered as a heatmap in which some portion having high contribution to classification is colored in red, for example, it is possible to determine the likelihood of classification being accurate upon determining the ratio of red color occupying the heatmap.

Alternatively, it is possible to determine the likelihood of classification being accurate by determining the extent of similarity in which the contribution-presentation image is likely to be similar to a typical example of a contribution-presentation image for a disease class.

Using an assignment method for assigning microRNA types to elements of a matrix representing image-rendition data according to a sequence of five bases through nine bases selected from among nine bases of the microRNA 5′-terminal for each microRNA type, the imaging unit 182 may calculate values of elements of a matrix representing image-rendition data based on expression levels of microRNAs for each microRNA type assigned to elements of a matrix.

As to characteristics of microRNAs, in particular, a sequence of five bases through nine bases of the microRNA 5′-terminal has a high impact of affection. As a sequence of bases, for example, it is possible to use a sequence of seven bases. Using the aforementioned assignment method, the imaging unit 182 can assign types of microRNAs having similar characteristics in an expression-level-data image to neighboring elements in a matrix. Since the contribution-presentation-image generation unit 193 generates an image such as a heatmap-style image, it is possible to visually grasp the grounds of classification indicated by the image or contributions of some portions of the input image to classification.

As the aforementioned assignment method, the imaging unit 182 may adopt an assignment method to assign microRNA types to pixels based on the Levenshtein distance relating to a sequence of five bases through nine bases selected from among nine bases of the microRNA 5′-terminal.

Accordingly, it is expected that microRNA types having similar characteristics in an expression-level-data image be disposed at neighboring pixels.

The display 120 may display a contribution-presentation image generated by the contribution-presentation-image generation unit 193 together with a typical contribution-presentation image of the class selected for image-rendition data.

Since any person looking at contribution-presentation images such as an extracted person can determine the extent of similarity between the presently-displayed contribution-presentation image and the typical contribution-presentation image of the class selected for image-rendition data, for example, it is possible to assess the degree of progression in diseases or the seriousness of diseases.

The classification unit 194 may classify the image-rendition data into the healthy class, disease classes for respective diseases, or a pre-symptomatic class of a specific disease which is set in association with at least one disease class relating to a specific disease among disease classes. For example, the image-rendition data can be rendered as an expression-level-data image.

Accordingly, it is possible for the image generation device 100 to distinctly present the healthy state, the sick state, and the pre-symptomatic state.

Instead of the image generation device 100 of FIG. 1 configured to generate and display a contribution-presentation image, it is possible to independently provide a device configured to present a contribution-presentation image and another device configured to display the contribution-presentation image.

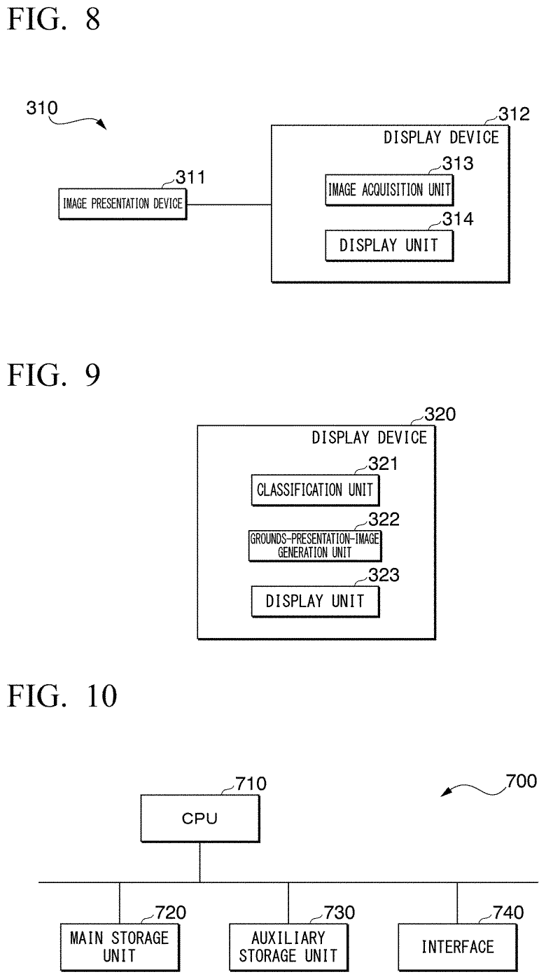

FIG. 8 is a block diagram showing a first configuration example of a display device according to another embodiment. In the configuration shown in FIG. 8, a display system 310 includes an image presentation device 311 and a display device 312. The display device 312 includes an image acquisition unit 313 and a display unit 314.

In the above configuration, the image presentation device 311 is configured to transmit a contribution-presentation image to the display device 312. As described above, the contribution-presentation image represents a contribution of some portion of image-rendition data in classification of image-rendition data, which can be produced by converting data representing an expression level for each microRNA type. In this connection, the image presentation device 311 may generate a contribution-presentation image according to a similar method of the image generation device 100. Alternatively, the image presentation device 311 may store a contribution-presentation image, which was already generated, so as to transmit the contribution-presentation image to the display device 312.

In the display device 312, the image acquisition unit 313 is configured to acquire the contribution-presentation image from the image presentation device 311. Specifically, the image acquisition unit 313 may receive data from the image presentation unit 311 so as to extract a contribution-presentation image from the received data.

The display unit 314 is configured to display the contribution-presentation image acquired by the image acquisition unit 313.

In the above, it is possible to separately locate the image presentation device 311 and the display device 312 in different countries.

In FIG. 1, the imaging unit 182 is not essential to the image generation device 100.

FIG. 9 is a block diagram showing a second configuration example of a display device according to a further embodiment. According to the configuration shown in FIG. 9, the display device 320 includes a classification unit 321, a grounds-presentation-image generation unit 322, and a display unit 323.

In the above configuration, the classification unit 321 is configured to classify expression-level data. As described above, the expression-level data represents an expression level for each microRNA type.

The grounds-presentation-image generation unit 322 is configured to generate a grounds-presentation image. The grounds-presentation image is used to present a two-dimensional image representing the grounds of classification by the classification unit 321. As the grounds-presentation image, the ground-presentation-image generation unit 322 may generate a two-dimensional image visualizing a difference between the feature quantity, which is extracted from expression-level data, and the basis of classification employed by the classification unit 321.

The display unit 323 is configured to display the grounds-presentation image generated by the ground-presentation-image generation unit 322.

FIG. 10 is a block diagram showing the configuration of a computer according to at least one embodiment. According to the configuration shown in FIG. 10, a computer 700 includes a CPU (Central Processing Unit) 710, a main storage device 720, an auxiliary storage device 730, and an interface 740.

At least one or more of the foregoing image generation device 100, the display device 312, and the display device 320 can be mounted on the computer 700. In this case, a series of processes realized by the foregoing processing parts are stored on the auxiliary storage device 730 in the form of programs. The CPU 710 may read programs from the auxiliary storage device 730, deploy programs on the main storage unit 720, and execute the foregoing processes according to programs. In addition, the CPU 710 may secure various storage areas corresponding to the foregoing storage units on the main storage device 720. For example, the auxiliary storage device 730 can be configured of non-volatile (or non-transitory) storage media such as CDC (Compact Disc) and DVD (Digital Versatile Disc).

When the image generation device 100 is mounted on the computer 700, the functionality of the control unit 180 and its related operations are stored on the auxiliary storage device 730 in the form of programs. The CPU 710 may read programs from the auxiliary storage device 730, deploy programs on the main storage device 720, and execute the foregoing processes according to programs.

In addition, the CPU 710 may secure storage areas corresponding to the storage unit 710 on the main storage device 720 according to programs.

The interface 740 provided with a communication function may realize a communication conducted between the communication unit 110 and another device under the control of the CPU 710. The interface 740 provided with a display device to display images may realize the function of the display unit 120 under the control of the CPU 710. The interface 740 provided with an input device to receive a user operation and to output a signal representing the user operation to the CPU 710 may realize the function of the operation-input unit 130.

When the display device 312 is mounted on the computer 700, for example, it is possible to realize the function of the image acquisition unit 313 by means of the CPU 710 configured to control the communication function of the interface 740 according to programs. It is possible to realize the function of the display unit 314 by means of the CPU 710 configured to control the display screen of the interface 740 according to programs.

When the display device 320 is mounted on the computer 700, the processing of the classification unit 321 and the processing of the grounds-presentation-image generation unit 322 are stored on the auxiliary storage device 730 in the form of programs. The CPU 710 reads programs from the auxiliary storage device 730 to deploy programs on the main storage device 720, thus executing their processing according to programs. The functionality of the display unit 323 can be realized by the CPU 710 executing programs for the display screen installed in the interface 740.

In this connection, it is possible to store on computer-readable storage media programs for performing the entirety of or some of the functions relating to the control unit 180, the display device 312, and the display device 320, wherein a computer system may load and execute programs stored on storage media so as to achieve the aforementioned functions of various parts. Herein, the term “computer system” may embrace OS (Operating System) and hardware such as peripheral devices.

In addition, the term “computer-readable storage media” refers to flexible disks, magneto-optical disks, ROM (Read-Only Memory), portable media such as CD-ROM (Compact-Disk Read-Only Memory), or storage devices such as hard disks embedded in computer systems. The aforementioned programs may achieve some of the foregoing functions, or the aforementioned programs may achieve the foregoing functions when combined with the existing programs stored on computer systems in advance.

Heretofore, the foregoing embodiments of the present invention have been described in detail with reference to the accompanying drawings, although concrete configurations are not necessarily limited to the foregoing embodiments; hence, the present invention may include any design change not departing from the subject matter of the invention.

### INDUSTRIAL APPLICABILITY

The present invention can be applied to image generation devices, display devices, data conversion devices, image generation methods, presentation methods, data conversion methods, and programs.

### REFERENCE SIGNS LIST

100 image generation device

110 communication unit

120, 314, 323 display unit

130 operation-input unit

170 storage unit

180 control unit

181 expression-level-data acquisition unit

182 imaging unit

190 visualizing unit

191 feature-quantity extraction unit

192 weight calculation unit

193 contribution-presentation-image generation unit

194, 321 classification unit

195 machine-learning control unit

211 encoder

212 decoder

213 multiplier

214 averaging operator

215 Argmax operator

310 display system

311 image presentation device

312, 320 display device

313 image acquisition unit

322 grounds-presentation-image generation unit

1. An image generation device comprising:
an imaging unit configured to convert data representing an expression level for each microRNA type into image-rendition data serving as data representing a matrix of two dimensions or more;
a classification unit configured to perform classification of the image-rendition data; and
a contribution-presentation-image generation unit configured to generate a contribution-presentation image representing a contribution of a specific part of the image-rendition data to the classification.

2. The image generation device according to claim 1, wherein according to an assignment method for assigning microRNA types to elements of a matrix representing the image-rendition data according to a sequence of five bases through nine bases selected from among nine bases for each microRNA 5′-terminal, the imaging unit is configured to calculate values of the elements of the matrix representing the image-rendition data based on expression levels of the microRNA types assigned to the elements of the matrix.

3. The image generation device according to claim 2, wherein the imaging unit adopts the assignment method for assigning the microRNA types to the elements of the matrix according to a Levenshtein distance relating to the sequence of five bases through nine bases selected from among nine bases for each microRNA 5′-terminal.

4. The image generation device according to claim 1, further comprising:
a display unit configured to display the contribution-presentation image generated by the contribution-presentation-image generation unit together with a typical contribution-presentation image in a class selected for the image-rendition data.

5. The image generation device according to claim 1, wherein the classification unit is configured to classify the image-rendition data into any one of a healthy class, a disease class for each disease, and a pre-symptomatic class for a specific disease provided for at least one disease among diseases.

6. A display device comprising:
a contribution-presentation-image acquisition unit configured to acquire a contribution-presentation image representing a contribution of a specific part of image-rendition data to classification of the image-rendition data which is produced by converting data representing an expression level for each microRNA type; and
a display unit configured to display the contribution-presentation image.

7. A display device comprising:
a classification unit configured to perform classification of data representing an expression level for each microRNA type;
a contribution-presentation-image generation unit configured to generate a grounds-presentation image presenting grounds of the classification in a two-dimensional image; and
a display unit configured to display the grounds-presentation image.

8. A data conversion method comprising:
rendering data representing an expression level for each microRNA type with a matrix of two dimensions of more; and
converting the data into image-rendition data, in which microRNA types having a smaller distance defined between index values corresponding to the microRNA types are assigned to neighboring elements in the matrix.

9. The data conversion method according to claim 8, further comprising:
calculating values of the elements of the matrix representing the image-rendition data based on expression levels of the microRNA types assigned to the elements of the matrix according to an assignment method for assigning microRNA types to elements of the matrix representing the image-rendition data according to a sequence of five bases through nine bases selected from among nine bases for each microRNA 5′-terminal.

10. The data conversion method according to claim 9, wherein the assignment method is configured to assign the microRNA types to the elements of the matrix according to a Levenshtein distance relating to the sequence of five bases through nine bases selected from among nine bases for each microRNA 5′-terminal.

11. (canceled)

12. A presentation method comprising:
performing classification of data representing an expression level for each microRNA type extracted from an extracted person;
generating a grounds-presentation image for presenting grounds of the classification in a two-dimensional image; and
displaying the grounds-presentation image to be presented to the extracted person.

13. (canceled)

14. (canceled)

15. (canceled)
